(19) United States
(12) Patent Application Publication
(10) Pub. No.: US 2015/0119740 A1
(43) Pub. Date: Apr. 30, 2015

(54) WHOLE-BODY PLETYSMOGRAPHY SYSTEM FOR THE CONTINUOUS CHARACTERIZATION OF SLEEP AND BREATHING IN A MOUSE

(71) Applicants: Adam Breaux Hernandez, Baltimore, MD (US); Jason Kirkness, Baltimore, MD (US); Hartmut Schneider, Lutherville, MD (US); Mikhael Polotsky, Baltimore, MD (US); Alan Schwartz, Baltimore, MD (US); Philip Smith, Baltimore, MD (US); Walter Hernandez, Potomac, MD (US)

(72) Inventors: Adam Breaux Hernandez, Baltimore, MD (US); Jason Kirkness, Baltimore, MD (US); Hartmut Schneider, Lutherville, MD (US); Mikhael Polotsky, Baltimore, MD (US); Alan Schwartz, Baltimore, MD (US); Philip Smith, Baltimore, MD (US); Walter Hernandez, Potomac, MD (US)

(73) Assignee: THE JOHNS HOPKINS UNIVERSITY, Baltimore, MD (US)

(21) Appl. No.: 14/129,556

(22) PCT Filed: Jul. 2, 2012
(86) PCT No.: PCT/US2012/045223

§ 371 Date: May 2, 2014

Related U.S. Application Data
(60) Provisional application No. 61/503,256, filed on Jun. 30, 2011.

(51) Int. Cl. A61B 5/08; A61B 5/0488; A61B 5/00; A61B 5/0476

(52) U.S. Cl. CPC A61B 5/0806; A61B 5/0476; A61B 5/0488; A61B 5/7203
USPC 600/529; 600/301

(57) ABSTRACT

An embodiment in accordance with the present invention provides a device for whole-body plethysmography (WBP) of a mouse for the continuous characterization of sleep and breathing. The inherent limitations of standard WBP are addressed to enable the continuous recording of validated measures of tidal volume, tidal airflow, and respiratory effort surrogate in an unrestrained, unanesthetized mouse. The addition of standard EEG and EMG recording technology allows for respiratory patterns to be fully characterized during sleep and wakefulness. The present invention also allows for the demonstration of the development of dynamic upper airway obstruction [inspiratory flow limitation (IFL)] during sleep in a susceptible, obese murine strain.

### CROSS REFERENCE TO RELATED APPLICATIONS

This application claims the benefit of U.S. Provisional Patent Application No. 61/503,256, filed Jun. 30, 2011, which is incorporated by reference herein, in its entirety.

### GOVERNMENT SUPPORT

This invention was made with government support under HL50381, HL37379, T3290030860 awarded by the National Institute of Health. The government has certain rights in the invention.

### FIELD OF THE INVENTION

The present invention relates generally to the study of respiration and sleep. More particularly, the present invention relates to a device for whole-body plethysmography of a mouse for the continuous characterization of sleep and breathing.

### BACKGROUND OF THE INVENTION

Sleep is associated with marked alterations in ventilatory control that lead to perturbations in breathing patterns, frank periods of hypoventilation, and upper airway obstruction. In susceptible individuals, these changes precipitate a spectrum of sleep-related breathing disorders (SRBD) including obstructive sleep apnea (OSA) and hypoventilation syndromes. SRBD are highly prevalent in Western society and have been linked to numerous clinical sequelae, including stroke, hypertension, diabetes, and frank respiratory failure. In humans, the polysomnogram, which encompasses respiratory (tidal volume, airflow, and effort) and electroencephalographic (EEG)/electromyographic (EMG) signals, is a pivotal tool for characterizing SRBD and sleep-related respiratory disturbances, including periodic apneas and hypopneas and alterations in gas exchange, tidal volume, and airflow.

Animal models can offer powerful insight into the pathogenesis of SRBD. Investigators have demonstrated recurrent central and obstructive apnea episodes with continuous recordings of airflow and respiratory effort in several large-animal models. Nevertheless, these studies have been hindered by difficulty in procuring, instrumenting, and acclimatizing the animals to polysomnographic recordings. Mice and rats have also been utilized to characterize ventilatory control and respiratory instability during sleep and wakefulness based on measures of tidal volume, respiratory rate, and minute ventilation. Mice are ideally suited for the study of SRBD mechanisms, since a wide variety of inbred strains are available, body weight and environment can be easily manipulated, and large numbers of animals can be rapidly characterized. In addition, obese strains susceptible to upper airway obstruction and SRDB have been identified. Nevertheless, measures of tidal airflow and respiratory effort have been lacking, limiting the ability to detect upper airway obstruction during sleep. In addition, methods for obtaining the full complement of continuous, high-fidelity tidal volume, tidal airflow, respiratory effort, and EEG/EMG signals in mice have not been developed.

Whole body plethysmography (WBP) has been utilized to record respiration during sleep and wake in unrestrained, freely moving mice. This approach provides an indirect measure of tidal volume, which is directly proportional to the cyclic chamber pressure signal produced during respiration in a sealed chamber. While the overall accuracy of WBP-derived tidal volume measurement (compared with direct pneumotachography) has been confirmed, several key limitations limit the use of WBP for continuously monitoring respiratory signals during sleep and wakefulness, as follows. First, traditional WBP methods provide intermittent high-fidelity tidal volume measurement, but techniques for recording a continuous, accurate tidal volume signal have not been validated in commercial chambers. Second, the indirectly measured WBP tidal airflow signal has not been validated. Third, methods for assessing respiratory effort noninvasively have not been developed or validated.

It would therefore be advantageous to provide an approach for making continuous high-fidelity respiratory recordings during sleep and wakefulness in mice.

### SUMMARY OF THE INVENTION

The foregoing needs are met, to a great extent, by the present invention, wherein in one aspect, a device for whole body plethysmography of a small mammal, includes a first sealed chamber having an outer wall defining an inner chamber configured for receiving the small mammal. The device includes a second sealed chamber coupled to the first sealed chamber via a pressure transducer. The second sealed chamber acts as a reference chamber for the first sealed chamber. A sensor bladder is disposed within the first sealed chamber and configured to transduce a mechanical pressure change associated with a breath taken by the small mammal. A reference bladder is configured to produce a signal for cancellation of noise a chamber pressure signal, and a pressure transducer couples the sensor bladder to the reference bladder, such that the resultant signal represents the respiration of the small mammal without the noise.

In accordance with an aspect of the present invention, the small mammal can be a mouse. The first sealed chamber has a slow leak of air out of the chamber, and the second sealed chamber also has a slow leak of air out of the chamber. The first sealed chamber is further configured to receive a lead for taking an EMG of the small mammal as well as a lead for taking an EEG of the small mammal. A source of pressurized air and a source of negative pressure are coupled to the first sealed chamber. A flow of approximately 150 mL/min of air moves through the first sealed chamber. A platform is disposed within the inner chamber of the first sealed chamber and configured for receiving the small mammal. The sensor bladder is disposed between the small mammal and the platform, and the reference bladder is disposed under the platform. A first high resistance element is disposed between the source of pressurized air and the first sealed chamber, and a second high resistance element is disposed between the source of negative pressure and the first sealed chamber. Noise is further characterized as a chamber pressure signal.

In accordance with another aspect of the present invention, a method of performing whole body plethysmography of a small mammal includes placing the small mammal in a first sealed chamber coupled to a second sealed chamber. The second sealed chamber acts as a reference to the first sealed chamber. An air flow is provided through the first sealed chamber. The method also includes transducing mechanical pressure changes associated with a breath taken by the small mammal into a respiratory signal for the small mammal, and cancelling noise in the respiratory signal for the small mammal.

In accordance with another aspect of the present invention cancelling noise further includes cancellation of a contaminating chamber pressure signal. Cancelling noise can also further include using a reference bladder. Transducing mechanical pressure changes includes using a sensor bladder to sense the mechanical pressure changes associated with the breath taken by the mouse. The method can also include sensing EMG and EEG signals from the small mammal.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a schematic view of a mouse WBP device according to an embodiment of the present invention.

FIG. 2 illustrates a representative recording of a WBP tidal volume and airflow signal validation trial, according to an embodiment of the present invention.

FIG. 3 illustrates a Bland-Altman plot of VT difference (tracheal VT-WBP VT) vs. gold standard tracheal VT in four mice, according to an embodiment of the present invention.

FIG. 4 illustrates an identity plot of WBP airflow vs. gold standard tracheal flow in a single mouse, according to an embodiment of the present invention.

FIG. 5 illustrates a representative recording of respiratory movement signal validation study, according to an embodiment of the present invention.

FIG. 6 illustrates recording sections of a WBP full polysomnographic study demonstrating respiratory waveforms during quiet wakefulness (left), non-rapid eye movement (NREM) sleep (middle), and rapid eye movement (REM) sleep (right) in one mouse, according to an embodiment of the present invention.

FIGS. 7A and 7B illustrate recording examples from a WBP study in an New Zealand obese mouse during sleep, according to an embodiment of the present invention.

FIG. 8 illustrates a flow chart of a method of providing a whole body plethysmograph of a mouse, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device for whole-body plethysmography (WBP) of a mouse for the continuous characterization of sleep and breathing. The inherent limitations of standard WBP are addressed to enable the continuous recording of validated measures of tidal volume, tidal airflow, and respiratory effort surrogate in an unrestrained, unanesthetized mouse. The addition of standard EEG and EMG recording technology allows for respiratory patterns to be fully characterized during sleep and wakefulness. The present invention also allows for the demonstration of the development of dynamic upper airway obstruction [inspiratory flow limitation (IFL)] during sleep in a susceptible, obese murine strain.

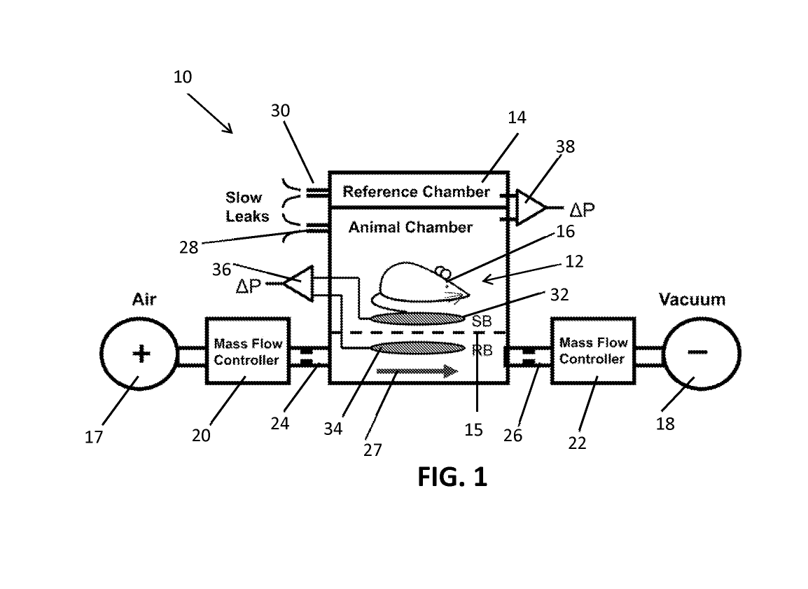

FIG. 1 illustrates a schematic view of a mouse WBP device according to an embodiment of the present invention. As illustrated in FIG. 1, the mouse WBP 10 includes a sealed animal chamber 12, a reference chamber 14, and a platform 15 to support the mouse 16. The animal chamber 12 is equipped with two ports (pneumotachographs) (not illustrated) on the upper surface and one large side port (not illustrated) and three small side ports (not illustrated) at the base. Positive and negative pressure sources 17, 18, respectively are utilized in series with mass flow controllers 20, 22 and high-resistance elements 24, 26 to generate a continuous bias flow 27 through the animal chamber 12 while maintaining a sufficiently high time constant. The high-resistance elements are interposed at the chamber's inflow and outflow ports so as to increase the time constant of the chamber to approximately 10 times that of the mouse's inspiratory time, thereby allowing for continuous, unattenuated signal recordings in an open system. The WBP chamber has a targeted time constant of 1.5 s (10 times the upper limit of a mouse's inspiratory time of ˜0.15 s). High-resistance elements (needles) were placed at the bias flow inlet and outlet to achieve this time constant.

The reference chamber, illustrated in FIG. 1 is configured to filter ambient noise from the pressure signal. Slow leaks 28, 30 present on both the animal chamber 12 and the reference chamber 14 allowed for equilibration with atmospheric pressure. A sensor bladder (SB) 32 is configured to transduce the mechanical pressure changes associated with mouse breathing, while a reference bladder (RB) 34 produces a signal that allows for cancellation of the contaminating chamber pressure signal via a differential pressure transducer 36.

The Drorbaugh and Fenn equation is used to calculate the WBP tidal volume signal from the WBP chamber pressure signal. Application of this formula requires the measurement of the following variables during each WBP recording session: mouse rectal temperature, chamber temperature, room temperature and relative humidity, and chamber gas constant, which is calculated by utilizing a known volume injection and the resultant WBP pressure deflection. In order to perform an exemplary, full polysomnographic recording session, using the device illustrated in FIG. 1, the chamber is humidified to approximately 90% relative humidity, and the mouse is allowed approximately 45 min to acclimate to the chamber before recordings are initiated. All signals are digitized at approximately 1,000 Hz (sampling frequency per channel) and recorded in LabChart 7 Pro (version 7.2). These specifications for a full polysomnographic recording session are merely provided as an exemplary embodiment, and any specifications known to or conceivable by one of skill in the art could be used.

WBP pressure can be recorded with a differential pressure transducer 38, illustrated in FIG. 1. The differential pressure transducer 38 is calibrated with a manometer against known pressures. The transducer 38 is activated by, and output to, an amplifier. A 0.5-Hz high-pass filter is also applied to the signal to remove low-frequency fluctuations around the baseline.

A first-order derivative (dV/dt) is applied to the WBP tidal volume signal to yield a WBP tidal airflow signal. The derivative can be calculated with a 25-point window, which acts as a low-pass filter and optimizes the signal-to-noise ratio of the WBP flow signal. Increasing the window width reduces noise, but results in attenuation of the high-frequency component (peak) of expiratory flow, while decreasing the window width results in increased noise. Optimization of window width is, therefore, performed by increasing the window width until the WBP flow signal had a satisfactory visual and graphic (R²>0.8) correlation with the tracheal flow signal.

Air bladders are positioned above (sensor bladder 32) and below (reference bladder 34) the rigid WBP platform, as illustrated in FIG. 1, such that they are completely isolated from each other. When the mouse 16 lays on the platform 15, the mechanical displacement of its torso during breathing is transduced by the upper sensor bladder 32, which is situated between the platform 15 and mouse 16. The signals from the sensor and reference bladders 32, 34 are compared with a differential pressure transducer 36, which subtracts the chamber pressure fluctuations in the reference bladder from the composite (chamber and motion pressure fluctuations) in the signal from sensor bladder 32. The approach effectively removes the contaminating tidal fluctuations in chamber pressure from the respiratory effort signal in the sensor bladder 32. In an exemplary embodiment, the air bladders 32, 34 are injected with approximately 0.5 ml of air and connected through the lateral base ports in the WBP chamber to a calibrated differential pressure transducer, which outputs to an amplifier. A band-pass filter can also be applied from 0.5 to 10 Hz to optimize the signal-to-noise ratio of the differential pressure signal.

Headmount leads are connected to a preamplifier, which is attached to a preamplifier analog adapter and output to an amplifier. The mouse endotracheal tube is connected to a calibrated pneumotachograph (resistance=0.40 cmH₂O·s·ml⁻¹). The pneumotachograph is connected to a differential pressure transducer, output to an amplifier. The tracheal flow was integrated to yield a tidal volume signal.

A bias flow of 150 ml/min is adequate to maintain a level of CO₂<1% (mean 0.4%, range 0.2-0.9%). This flow is maintained by applying pressurized air 17 and a vacuum source 18 across the inlet and outlet of the chamber 12, respectively. Minor fluctuations in inflow and outflow rates, however, led to transient deviations in chamber pressure from atmospheric. To prevent such deviations, air delivery and removal are precisely regulated with mass flow controllers 20, 22 (inlet setting: mass flow=0.152, outlet setting: mass flow=0.152). The mass flow controllers 20, 22 allow for the inlet and outlet bias flows to be precisely matched by mass rather than volume, which changes across the high-resistance elements. Minor pressure fluctuations are further minimized by implementing a slow leak 28 through a needle connected to another chamber port to allow the chamber pressure to equilibrate with atmospheric pressure. After implementing the high-resistance elements and slow leak, the final time constant of the chamber is 1.6 s in duration.

To cancel out ambient barometric pressure changes and noise, the WBP animal chamber 12 is referenced to a reference chamber 14 with a similar time constant using a differential pressure transducer 38. Because of the increased time constant of the animal chamber 12, a high-resistance element in the form of a needle is also incorporated into the reference chamber slow leak port 30 to match the time constants of the two chambers. To check the overall resilience of the system to changes in ambient pressure, local atmospheric disturbances (e.g., opening and closing the laboratory door) were created, and in the experiment no deflection in the pressure signal was noted.

In an exemplary experiment, to compare the measurements of chamber pressure in the present invention to gold standard closed-system (sealed) chamber measurements, a small-mammal mechanical ventilator is connected to the WBP 10 to produce a cyclic pressure signal. The WBP pressure signal fluctuations are recorded serially in both the open- and closed-system configurations over ventilator frequencies ranging from 80 to 400 cycles/min, which encompass the upper and lower limits of mouse respiratory rates and inspiratory times (during both normal breathing and hypoxic and hypercapneic challenges). Pressure measurements in the open and closed system are compared to quantify any signal attenuation in the open system. At frequencies of 80-400 cycles/min, a flat frequency response is demonstrated in the open system that remained within 1% of that in the closed system, thus indicating no significant attenuation of the WBP pressure signal in the open system over the full range of potential mouse respiratory rates.

Further to the experiment, WBP measures of tidal volume and airflow in the present system were validated against gold standard pneumotachographic measurements of these variables in four mice. The recordings from the pneumotach and WBP were made simultaneously. Each mouse was anesthetized, intubated, and placed on a heating pad to maintain normal body temperature (to preserve the temperature gradient between the mouse and room air and thus maintain the respiratory-related changes in pressure). The endotracheal tube was connected to a calibrated pneumotachograph and differential pressure transducer. The mouse and pneumotachograph were then placed in the open-system WBP chamber (with bias flow applied). Simultaneous respiratory recordings were obtained from the pneumotachograph and WBP chamber for multiple 15- to 20-s periods, such that WBP and pneumotachographic measure of tidal volume and airflow could be compared on the same breaths. The mouse was taken out of the chamber and placed on the heating pad between recording periods. The mouse rectal temperature was taken before and after each recording period (differential ranged from 0 to 1.0° C.), and the average of these two numbers was used for calculating the WBP tidal volume. WBP measurements of calculated tidal volume and derived flow were compared with pneumotachographic measurement flow and tidal volume. The mouse was euthanized after the study.

A sensor air bladder and reference air bladder were placed in an open-system WBP, as described above with respect to FIG. 1. A small-mammal mechanical ventilator was attached to the WBP to simulate a cyclic pressure signal similar to that produced by a breathing mouse. The relative strength of the contaminating signal was checked both with and without a euthanized mouse placed on the sensor bladder to account for the potential impact of differences in the unstressed volume of the balloon on the effort signal. When the differential pressure transducer was referenced to atmospheric pressure, the mechanical ventilator produced a contaminating signal of ˜0.3 cmH₂O. When the differential pressure transducer was reconnected to the reference bladder, the contaminating signal was not detectable, whether or not the euthanized mouse was placed on the sensor cuff.

The respiratory movement signal was also validated against a gold standard pneumotachographic measure of tracheal pressure in three anesthetized mice. Each mouse was intubated and placed on the sensor air bladder (outside of the WBP chamber). The endotracheal tube was connected to a pneumotachograph and calibrated pressure transducer (no. P23, Gould Statham, Bayamon, PR). To obtain a wide range of tracheal pressures, dead space (45-cm tube, volume=2.8 cm³) were added to the breathing circuit for multiple 10- to 15-s periods in each mouse. As CO₂ accumulated in the breathing circuit, respiratory effort increased, and the differential bladder pressure was compared with the tracheal pressure signal. Mouse position and orientation on the air bladder were also varied with each trial to investigate a possible positional component to performance.

The tidal volume, airflow, respiratory movement, and EEG/EMG signals were acquired simultaneously and recorded continuously in an unrestrained C57BL/6J or NZO mouse in the open WBP system with a bias flow of 150 ml/min. After an equilibration period of 45 min, 2 h of recording were collected from 2 PM to 4 PM. Tidal volume, respiratory rate, and minute ventilation were measured during stable periods of wakefulness, non-rapid eye movement (NREM) and rapid eye movement (REM) sleep.

Values in the RESULTS section are reported as means and SDs. Correlation analysis was used to compare experimental measures (e.g., WBP tidal volume) to gold standard measures (e.g., tracheal tidal volume) in validation protocols. In addition, Bland-Altman analysis was utilized to quantify bias and limits of agreement (LOA). As previously described, a repeated-measures ANOVA was utilized to account for multiple measurements made within a single subject (3). A P value of <0.05 was accepted as the threshold for inferring statistical significance. Ninety-five percent LOA were calculated as ±1.96× the SD.

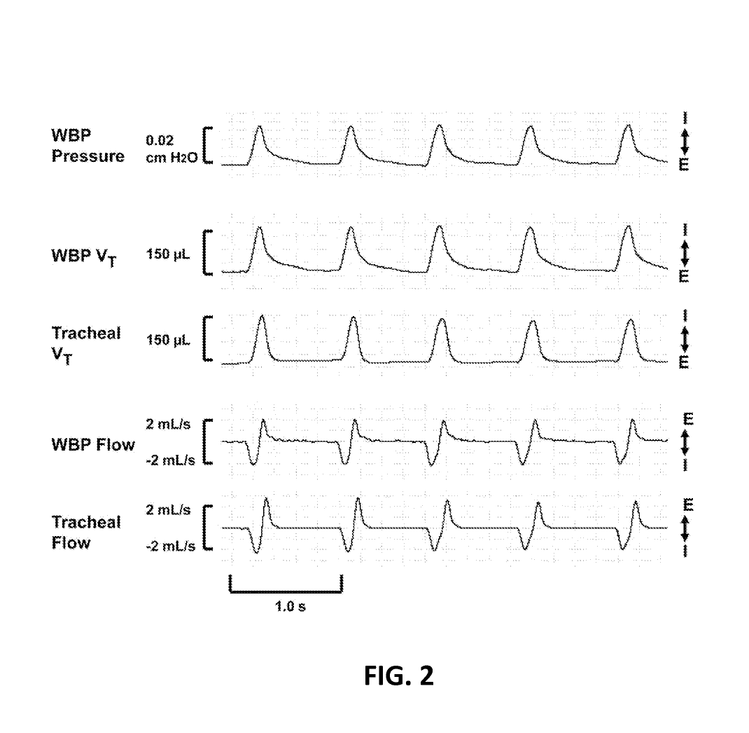

FIG. 2 illustrates a representative recording of a WBP tidal volume and airflow signal validation trial, which shows that the indirectly measured WBP tidal volume signal was similar in amplitude and morphology to the simultaneously obtained gold standard tracheal tidal volume signal during the inspiratory limb. The expiratory limb demonstrated a gradual roll-off or shoulder immediately before returning to baseline, consistent with previous reports. FIG. 2 further illustrates a representative recording of a tidal volume (Vt) and airflow validation study recording in a single, anesthetized mouse. WBP Vt was similar in amplitude and waveform morphology to tracheal Vt during the inspiratory limb and demonstrated a gradual roll-off or shoulder before returning to baseline in the expiratory limb. Compared with tracheal airflow, WBP inspiratory flow (I) showed a similar amplitude and morphology, while expiratory flow (E) demonstrated an attenuation in signal amplitude but similar morphology. Signals (from top to bottom) include WBP pressure, WBP Vt, tracheal Vt, WBP airflow, and tracheal airflow.

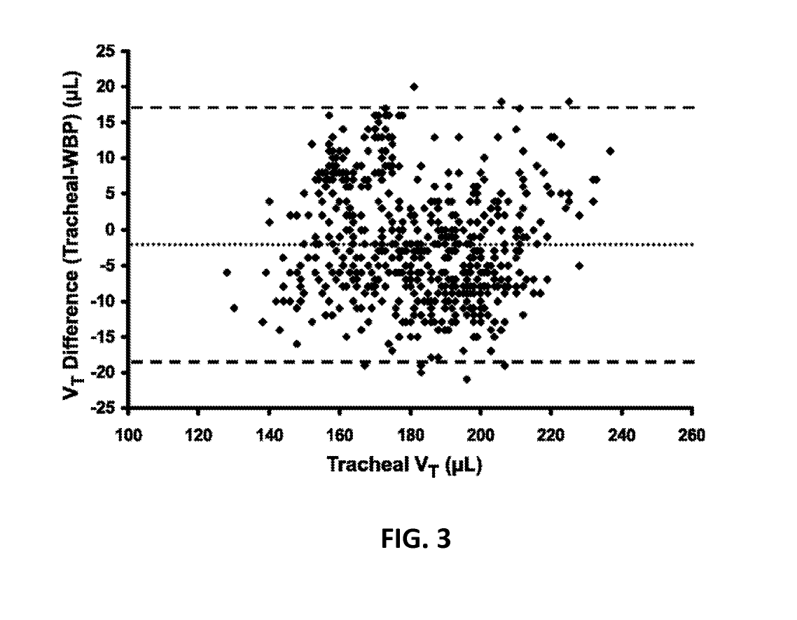

FIG. 3 illustrates a Bland-Altman plot that was used to compare WBP tidal volume (for all four mice) to tracheal tidal volume. Tracheal tidal volume was plotted on the X-axis (rather than mean tidal volume) as the gold standard. The mean difference of the tidal volume signals (−1.80 μl) represented only 1% of the mean tracheal tidal volume, indicating minimal systematic bias in the WBP signal. The 95% LOA were also narrow (±17.50 μl around the mean difference), which represented ˜10% of mean tracheal tidal volume. No skew was apparent in mean tidal volume difference as a function of change in tracheal tidal volume over a wide range of tidal volumes. Bland-Altman analysis of the individual mouse trials (Table 1, top; plots not shown) revealed minimal bias, no skew, and within-mouse LOA of ±10.88 μl (SD 5.55 μl, P<0.001), which were even more narrow than those for the combined data. The correlation coefficient (R²) between tracheal tidal volume and WBP tidal volume of the combined data was 0.87 (P<0.001). Further, FIG. 3 illustrates a Bland-Altman plot of VT difference (tracheal VT-WBP VT) vs. gold standard tracheal VT in four mice. The Y-axis represents the difference between the tracheal and plethysmographic VT values. The dotted line delineates the mean VT difference, which is −1.80 μl. The limits of agreement lie at 15.7 and −19.3 μl (±17.5 μl) (SD 8.9 μl, P<0.001). There was no skew as a function of increasing or decreasing tracheal VT.

TABLE 1

| | | | | | | | |
|---|---|---|---|---|---|---|---|
| | | | | | Mean | | |
| | No. of | Mean | Mean | SD, | Difference | Relative Mean | |
| Mouse | Breaths | V_{T}, μl | Difference, μl | μl | 95% CI | Difference, % | CV, % |

V_{T}

| 1 | 142 | 193.2 | 0.0 | 7.2 | −1.2-1.2 | 0.0 | 3.7 |
|---|---|---|---|---|---|---|---|
| 2 | 189 | 180.4 | −6.2 | 4.8 | −6.9-5.5 | −3.5 | 2.7 |
| 3 | 131 | 163.8 | 7.9 | 4.8 | 7.1-8.8 | 4.8 | 3.0 |
| 4 | 102 | 195.9 | −8.6 | 4.4 | −9.5-7.7 | −4.4 | 2.3 |
| Total | 564 | 182.6 | −1.8 | 8.9 | −2.5-1.1 | −1.0 | 4.9 |
| | No. of | | | | | | |
| | Breaths | Mean | Mean | | | | |
| | (5 Per | Flow, | Difference, | | Mean | Relative | CV, |
| Phase | Mouse) | μl/s | μl/s | SD, μl/s | Difference | Mean | % |

Tidal airflow

| | | | | | | | |
|---|---|---|---|---|---|---|---|
| Inspiration | 20 | 845.1 | −40.5 | 264.9 | −49.5-31.5 | 3.7 | 24.4 |
| Expiration | 20 | −761.8 | −201.7 | 698.5 | −219.4-184 | −30.9 | |

FIG. 2 shows a recording of the WBP airflow signal adjacent to the simultaneously obtained gold standard tracheal airflow signal. Compared with the tracheal flow signal, the WBP inspiratory flow waveform was similar in amplitude and morphology, while expiratory flow showed an attenuation in signal amplitude but similar morphology.

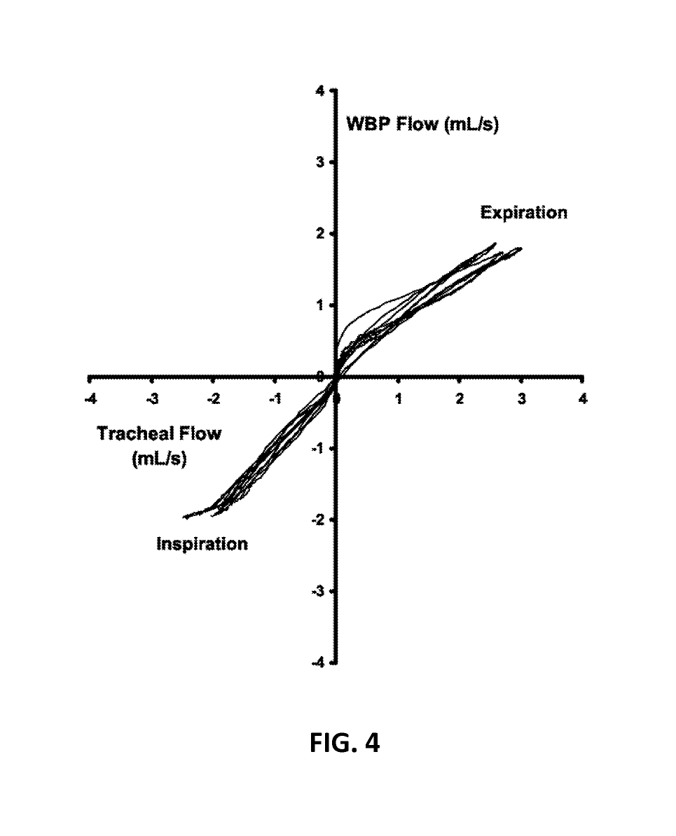

FIG. 4 illustrates a comparison of the WBP and tracheal flow signals for the five representative breaths illustrated in the recording example in FIG. 2. This graph reveals a high degree of correlation throughout the respiratory cycle between the two (R²=0.97 for inspiration, R²=0.96 for expiration, P<0.001 for both). Specifically, the WBP and tracheal airflow signals tracked one another along the line of identity throughout inspiration, as illustrated in the bottom left quadrant of FIG. 4. In contrast, while WBP expiratory flow correlated well with tracheal flow, attenuation of the WBP flow signal was evident as tracheal expiratory flow increased, as illustrated in the skew in curve at higher tracheal flow levels at the right top quadrant of FIG. 4. Further, FIG. 4 illustrates an identity plot of WBP airflow vs. gold standard tracheal flow in a single mouse, as illustrated in FIG. 2. The X-axis represents tracheal airflow, and the Y-axis represents WBP airflow. Positive flow indicates expiration, and negative flow indicates inspiration. Good correlation was seen across both the inspiratory (R²=0.97, P<0.001) and expiratory (R²=0.96, P<0.001) phases. Inspiratory flow demonstrated good agreement between WBP and tracheal values, while expiratory flow showed attenuation of the WBP flow relative to the tracheal flow signal.

In Table 1, above, separate Bland-Altman analyses appear for inspiratory and expiratory flow data encompassing four mice (five breaths each). Inspiratory data showed good agreement between WBP inspiratory flow and the gold standard (relative mean difference=3.7%). Expiratory phase data demonstrated significant attenuation of WBP expiratory flow compared with tracheal flow (relative mean difference=−30.9%). Bland-Altman analyses (plots not shown) of our flow data yielded LOA of ±519.2 μl (SD 264.9, P<0.001) for inspiratory flows and ±1,369.1 μl (SD 698.5, P<0.001) for expiratory flows. The correlation coefficients (R²) of the inspiratory and expiratory phases of the combined data were 0.92 (P<0.001) and 0.83 (P<0.001), respectively.

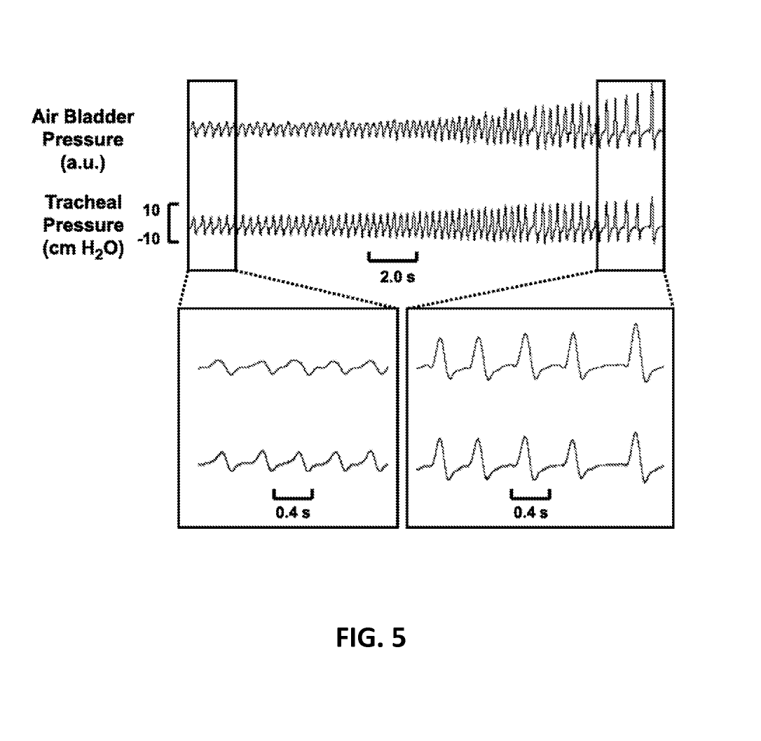

As illustrated in FIG. 5, representative recordings of noninvasive respiratory movement (air bladder pressure) and gold standard tracheal pressure illustrate the response of these signals to CO₂ rebreathing when dead space was added to the breathing circuit of an anesthetized mouse. As shown, the air bladder pressure swings tracked those in the tracheal pressure as effort progressively increased, as illustrated at the top of FIG. 5. Expanded views at low, as illustrated in FIG. 5 at the bottom left and high, as illustrated in FIG. 5 on the bottom right, demonstrate that excursions in the air bladder pressure paralleled those in the tracheal pressure signal. Further, FIG. 5 illustrates a representative recording of respiratory movement signal validation study. The top signal represents air bladder pressure (novel respiratory effort signal), and the bottom signal represents tracheal pressure (gold standard respiratory effort signal). During the rebreathing trial, both tracheal pressure and air bladder pressure increased in parallel (top panel). Expanded recordings at low (bottom left panel) and high (bottom right panel) effort further demonstrate that the two signals paralleled one another over a wide range of effort.

To further investigate the correlation between these two signals, peak tidal swings (amplitudes) in air bladder pressure and tracheal pressure were compared in each of three mice for a total of 17 rebreathing trials and 908 breaths. In these mice, mean correlation coefficients (R²) were 0.79 (0.60-0.86), 0.84 (0.76-0.93), and 0.84 (0.81-0.92) (all P values<0.001), demonstrating a high degree of correlation in the amplitude of the respiratory movement signal and tracheal pressure signal across multiple trials. We did not detect any influence of mouse position or orientation on the air bladder on signal performance, as reflected by the high level of correlation between signals.

To further characterize the performance characteristics of the air bladder respiratory movement signal, within-breath excursions of the air bladder pressure signal were correlated to gold standard tracheal pressure signal over five breaths in each mouse during the rebreathing trials. Correlation coefficients (R²) in the three mice were 0.96, 0.86, and 0.93 (all P values<0.001), indicating that the air bladder-transduced respiratory effort signal correlated closely with tracheal pressure over the full waveform of each breath.

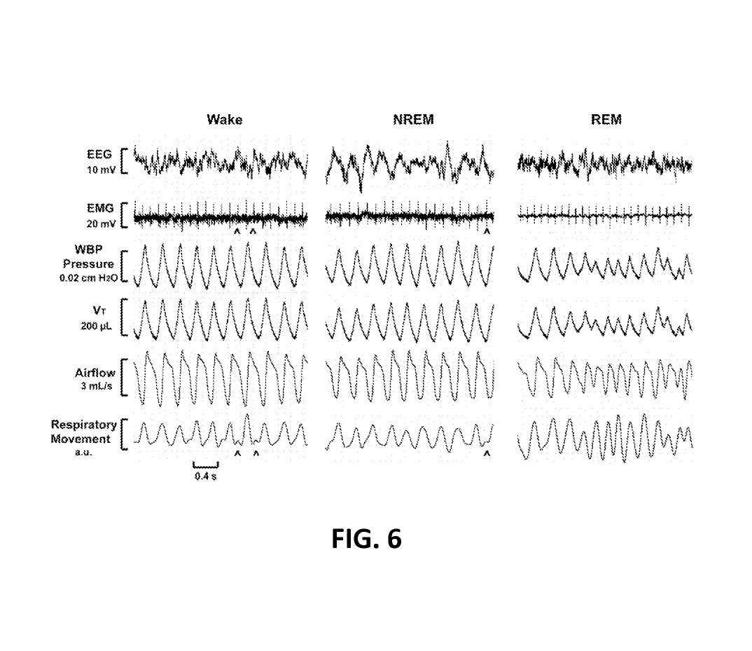

FIG. 6 shows recording segments from a 2-h full polysomnographic study from one mouse. Quiet wakefulness was characterized by a respiratory pattern that was regular in amplitude and timing. Mean tidal volume, respiratory rate, and minute ventilation were 240 μl (range=220-255 μl), 217 breaths/min, and 52.1 ml/min, respectively. NREM sleep demonstrated a regular respiratory pattern with a mean tidal volume, respiratory rate, and minute ventilation of 217 μl (range=198-230 μl), 211 breaths/min, and 45.8 ml/min, respectively. REM sleep was characterized by an irregular breathing pattern with highly variable tidal volumes. Mean tidal volume, respiratory rate, and minute ventilation were 125 μl (range=66-196 μl), 289 breaths/min, and 36.1 ml/min, respectively. REM sleep demonstrated a period of decreasing tidal volumes with simultaneously increasing respiratory movement, which may indicate an increase in airway resistance during that period.

Further, FIG. 6 illustrates recording sections of a WBP full polysomnographic study demonstrating respiratory waveforms during quiet wakefulness (left), non-rapid eye movement (NREM) sleep (middle), and rapid eye movement (REM) sleep (right) in one mouse. Signals (from top to bottom) include electroencephalographic (EEG) signal, nuchal electromyographic (EMG) signal, WBP chamber pressure, WBP VT, WBP airflow, and respiratory movement (surrogate for respiratory effort). Intermittent cardiac artifact (carets) can be seen in the EMG and respiratory movement channels.

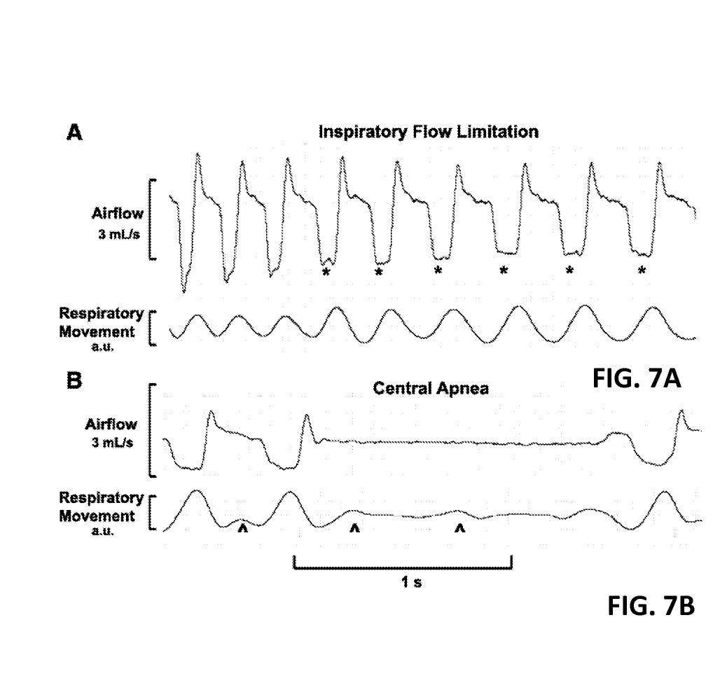

During a 2-h full polysomnographic study in a single NZO mouse, several sleep-related disturbances in airflow were observed, as illustrated in the representative examples of FIG. 7. FIG. 7A shows a period of progressive decreases in inspiratory airflow. In the first several breaths, a somewhat rounded inspiratory flow contour gave way to broader plateaus in early inspiration on subsequent breaths. Inspiratory airflow plateaued at a maximal level (maximal inspiratory airflow), despite increasing inspiratory respiratory movement (see asterisks), suggesting the presence of IFL. These breaths also exhibited other characteristics of IFL, including reductions in maximal inspiratory airflow, increased respiratory movement swings, increased inspiratory time, and negative effort dependence. In contrast, flow and effort signal fluctuations ceased during a 1.4-s central apnea, as illustrated in FIG. 7B. FIGS. 7A and 7B illustrate recording examples from a WBP study in an New Zealand Obese mouse during sleep. FIG. 7A illustrates a period of progressive decrease in inspiratory airflow. The inspiratory flow contour exhibited progressively broader midinspiratory plateaus (see asterisks), despite increasing inspiratory respiratory movement, consistent with the development of inspiratory flow limitation (IFL). Additional characteristics of IFL include increased respiratory movement swings (compared with earlier breaths), a reduction in maximal inspiratory airflow, an increase in inspiratory time, and negative effort dependence (which can be seen on the first, third, and fifth flow-limited breaths). FIG. 7B illustrates a 1.4-s central apnea characterized by the absence of respiratory flow and movement. Intermittent cardiac artifact (see carets) can be seen in the effort signal.

Using the device illustrated in FIG. 1, sleep-related changes were detected in ventilatory pattern, as illustrated in FIG. 6, and disturbances in respiratory airflow, as illustrated in FIGS. 7A and 7B. Of note, airflow and respiratory movement signals were crucial in detecting IFL, which is characterized by increasing respiratory effort during a plateau in inspiratory airflow, in an obese mouse strain previously considered susceptible to upper airway obstruction. These flow-limited breaths were characterized by a decrease in inspiratory flow, an increase in inspiratory time, an increase in respiratory movement, and negative effort dependence, all of which are consistent with the development of dynamic upper airway obstruction. Previous studies have suggested that the most likely site of extrathoracic obstruction in the mouse is the pharynx. IFL in a sleeping mouse is a novel, potentially intriguing finding and suggests that this strain might serve as mouse model of OSA. While central apneas have been reported in both mice and rats, a rodent model of OSA has never been reported.

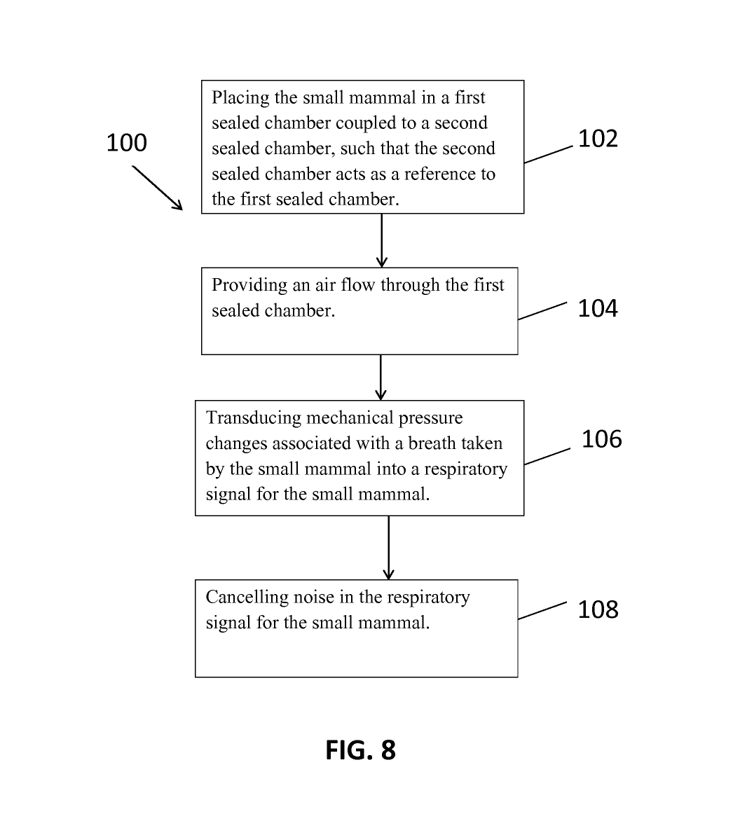

As illustrated in FIG. 8, a method of performing whole body plethysmography of a small mammal 100 includes a step 102 of placing the small mammal in a first sealed chamber coupled to a second sealed chamber. In step 102, the second sealed chamber acts as a reference to the first sealed chamber. In step 106, an air flow is provided through the first sealed chamber. The method also includes step 108 of transducing mechanical pressure changes associated with a breath taken by the small mammal into a respiratory signal for the small mammal, and step 110 of cancelling noise in the respiratory signal for the small mammal.

It should be noted that in certain embodiments, greater accuracy of the expiratory flow signal could be implemented by incorporating a correction factor, increasing the signal-to-noise ratio for the underlying chamber pressure, or signal averaging the expiratory signal over multiple breaths. Oxyhemoglobin saturation monitoring could also be incorporated. Additionally, while this system was developed solely to studying mice, it can be used for rat plethysmography with some modifications. A larger commercial chamber is necessary, and this chamber must be fitted with the proper needle resistances to achieve an adequate time constant and bias flow.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A device for whole body plethysmography of a small mammal, comprising:
a first sealed chamber having an outer wall defining an inner chamber configured for receiving the small mammal;
a second sealed chamber coupled to the first sealed chamber via a pressure transducer, such that the second sealed chamber acts as a reference chamber for the first sealed chamber;
a sensor bladder disposed within the first sealed chamber and configured to transduce a mechanical pressure change associated with a breath taken by the small mammal;
a reference bladder configured to produce a signal for cancellation of noise a chamber pressure signal; and
a pressure transducer coupling the sensor bladder to the reference bladder, such that the resultant signal represents the respiration of the small mammal without the noise.

2. The device of claim 1 wherein the small mammal comprises a mouse.

3. The device of claim 1 further comprising the first sealed chamber being configured to have a slow leak of air out of the chamber.

4. The device of claim 1 further comprising the second sealed chamber being configured to have a slow leak of air out of the chamber.

5. The device of claim 1 further comprising configuring the first sealed chamber to receive a lead for taking an EMG of the small mammal.

6. The device of claim 1 further comprising configuring the first sealed chamber to receive a lead for taking an EEG of the small mammal.

7. The device of claim 1 further comprising a source of pressurized air being coupled to the first sealed chamber.

8. The device of claim 1 further comprising a source of negative pressure being coupled to the first sealed chamber.

9. The device of claim 1 further comprising a flow of approximately 150 mL/min of air through the first sealed chamber.

10. The device of claim 1 further comprising a platform disposed within the inner chamber of the first sealed chamber and configured for receiving the small mammal.

11. The device of claim 1 further comprising the sensor bladder being disposed between the small mammal and the platform.

12. The device of claim 1 further comprising the reference bladder being disposed under the platform.

13. The device of claim 7 further comprising a first high resistance element being disposed between the source of pressurized air and the first sealed chamber.

14. The device of claim 8 further comprising a second high resistance element being disposed between the source of negative pressure and the first sealed chamber.

15. The device of claim 1 wherein noise further comprises a chamber pressure signal.

16. A method of performing whole body plethysmography of a small mammal comprising:
placing the small mammal in a first sealed chamber coupled to a second sealed chamber, such that the second sealed chamber acts as a reference to the first sealed chamber;
providing an air flow through the first sealed chamber;
transducing mechanical pressure changes associated with a breath taken by the small mammal into a respiratory signal for the small mammal; and
cancelling noise in the respiratory signal for the small mammal.

17. The method of claim 16 wherein cancelling noise further comprises cancellation of a contaminating chamber pressure signal.

18. The method of claim 16 wherein cancelling noise further comprises using a reference bladder.

19. The method of claim 16 wherein transducing mechanical pressure changes further comprises using a sensor bladder to sense the mechanical pressure changes associated with the breath taken by the mouse.

20. The method of claim 16 further comprising sensing EMG and EEG signals from the small mammal.
